(19) United States
(12) Patent Application Publication
(10) Pub. No.: US 2020/0001108 A1
(43) Pub. Date: Jan. 2, 2020

(54) VARIABLE HIGH SPEED LASER TIP ADAPTER

(71) Applicant: LUMENIS LTD., YOKNEAM (IL)

(72) Inventors: Danny Koifman, Ganei Hatikva (IL); Ilan Shier, Binyamina (IL)

(21) Appl. No.: 16/571,124

(22) Filed: Sep. 15, 2019

Related U.S. Application Data
(63) Continuation of application No. PCT/IL2018/050406, filed on Apr. 8, 2018.
(60) Provisional application No. 62/482,841, filed on Apr. 7, 2017.

(51) Int. Cl. A61N 5/06

(52) U.S. Cl. CPC A61N 5/0616; A61N 2005/007

(57) ABSTRACT

An adapter tip for a light emitting device which has an open cavity with a set sized opening. The adapter fits with the opening and reduces the size of the opening from the set size to a size less than the set size to concentrate light emanating from the light emitting device. Further, the open cavity of the light emitting device includes a cooled transparent member which contacts and cools skin coming into contact with the cooled transparent member when the skin is drawn into the cavity under vacuum.

### RELATED APPLICATIONS

This application is a continuation of PCT/IL2018/050406, filed Apr. 8, 2018, which claims priority to U.S. Provisional Application No. 62/482,841, filed Apr. 7, 2017, the entire contents of which are incorporated herein by reference.

### FIELD OF THE PRESENT INVENTION

This invention relates to aesthetic treatment devices and in particular to laser treatment devices with variable size contact surfaces and with cooling.

### BACKGROUND OF THE PRESENT INVENTION

In the real world, oftentimes one size does not fit all. This is true in the field of aesthetic treatment of patients' skin tissue. Many skin treatment devices incorporate a handpiece which includes a delivery device. This delivery device is often in the form of an applicator that contacts the skin tissue. Depending on the portion of the body or even the size of the body part being treated, different sized applicators are desirable.

One type of known device that is made and sold by the assignee of the present invention, Lumenis LTD of Yokneam Israel is named the Lightsheer®. The Lightsheer® device includes an applicator in the form of a handpiece. Within the device is a source of laser light, and in this case the laser light is produced by an array of diodes, although the present invention is not limited to that source of energy. The handpiece also includes a patient contact portion, and in the case of a model called the Lightsheer® HS (for high speed), the portion of the handpiece engaging with the patient is in the form of a cavity into which the skin tissue is sucked under the influence of a vacuum force that draws the tissue into the cavity and draws it near the array of diodes, the array of diodes being mounted in the “bottom” of the cavity, so that when the tissue is sucked into the cavity, the tissue is placed near to the diode array.

Presently, the device described above is sold with a single size cavity and related aperture or opening which contacts the skin tissue. However, the present size aperture or opening may not fit in some areas of the body, such as around the eyes of in the groin area or in upper lip simply because it is too big compared to the target area or the skin laxicity is not enough to perform effective skin draw. Additional areas of treatment can include also bony areas such as in the tibia where vacuum is not always achieved as the bony area prevents good adhesion of the hand piece vacuum cavity to the tissue. In addition, sometimes it is desirable to provide more fluence or more energy per square mm to the skin tissue or improve the spot size homogeneity, for example to remove fine hair with low melanin concentration, hair removal being one of the main functions of the Lightsheer® device. Furthermore, it may be desirable to provide better spot beam profile homogeneity.

It is to resolve the above issues that a first embodiment of the present invention is directed, that is, to provide attachments or adapters to the Lightsheer® device to accommodate the desire to provide alternative sized apertures or openings in such a way that it is simple, does not involve electrical or other complications.

As mentioned, an aspect of the present Lightsheer® device is that the tissue is drawn close to the source of energy such as the laser diode array mounted in the “bottom” of the cavity.

However, the Lightsheer device is presently available with two handpieces, one being the HS handpiece as described above, the other being termed as the “ET” handpiece. These handpieces differ as follows. The HS handpiece, as mentioned, includes a cavity into which the skin tissue is sucked under vacuum control so that it enters the cavity and comes into close contact with the laser diode array at the “bottom” of the cavity. There is no skin cooling in the HS device, although cooling is provided for the diode array.

The ET handpiece does not include a cavity, but rather incorporates a sapphire tip at its distal end that receives energy from a laser diode array and is placed in contact with the skin tissue. The sapphire tip itself is cooled to reduce discomfort to the patient due to excessive heat being generated.

The ET sapphire has a smaller “footprint” and thus covers a smaller area of treatment than the HS handpiece. The HS has a larger “footprint”, has the benefit of a cavity into which the tissue is sucked, but does not provide cooling.

Thus, it would be advantageous to provide a device which incorporate the best attributes of both the ET and the HS. It is to this goal that this second embodiment of the present invention is directed.

### SUMMARY OF THE PRESENT INVENTION

In an aspect, an adapter tip for a light emitting device is disclosed; the light emitting device has a cavity, the cavity: being open on one end of size X, having depending side walls beginning at the open end and extending to a closed bottom wall; the closed bottom wall includes a light source which projects light from the closed bottom wall to and out the open end. The adapter includes: a housing having a plurality of joined side walls, the side walls terminating at two open ends; one of the open ends being sized at about the same size X as the open end of the cavity and attachable to the open end of the cavity; the other of the open ends being tapered from size X to a size Y, wherein Y is less than X in size; light projected from the light source is reduced from size X to size Y when emitted from the other of the open ends.

In a further aspect, the light emitting device further includes one or more apertures formed on the closed bottom wall, the one or more apertures being connectable to a vacuum source; the adapter further includes one or more tubes mounted on the open end of the adapter of about size X, the one or more tubes being insertable into the one or more apertures formed on the closed bottom wall, the one or more tubes operating to join the adapter to the light emitting device. A disposable insert is adapted for insertion into the other of the open ends in the adapter. The adapter may further include a plurality of adapters and wherein the other of the open ends in the plurality of adapters are of size Z, wherein Z is less than size Y.

In another aspect, a light emitting device for treating skin tissue has a housing, the housing having a cavity, the cavity being open on one end, a plurality depending side walls beginning at the open end and extending to a closed bottom wall, wherein the closed bottom wall includes a light source which projects light from the closed bottom wall to and out the open end; further, a solid transparent member mounted within the cavity, the member having two opposed faces and a plurality of side walls joining the two opposed faces; the member may be positioned in the cavity distally of the light source; a collar is mounted on and surrounding the plurality of side walls of the solid transparent member, the collar being constructed of a thermally conductive material; a cooling source operatively connected to the collar, the cooling source cooling the collar and the surrounded solid transparent member; a vacuum source, the vacuum source being operatively connected to one or more formed openings in the cavity; the vacuum source, when activated, draws skin tissue into the cavity to the extent that the skin tissue contacts the cooled solid transparent member; upon activation of the light source, heat is emitted from the light source, the skin tissue being cooled by the cooled solid transparent member.

In yet another aspect, the light source is one or more arrays of laser diodes and the cooling source is a cooled fluid, the collar including passages through which the cooled fluid enters and leaves the collar.

In yet a further aspect, the cooling source is a thermoelectric cooling device, the thermoelectric cooling device being mounted between the solid transparent member and the surrounding collar, further comprising one or more passages in the collar through which cooling fluid enters and leaves the collar.

In an aspect, a method of cosmetically treating the skin tissue, the steps comprising:

providing the disclosed light emitting device, then the steps of: placing the open end of the device onto the skin tissue; activating the vacuum source to thereby draw skin to draw the skin tissue into the cavity towards and in contact with a face of the solid transparent member; activating the cooling source to cool the solid transparent member; activating the light source, the light source causing light to travel from the light source through to the solid transparent member and to impinge on the skin tissue; as a result, the skin tissue is cooled by contact with the solid transparent member.

### BRIEF DESCRIPTION OF THE DRAWINGS

FIGS. 1A to 1D illustrate adapter tips of a first embodiment of the present invention.

FIGS. 2 to 4 illustrate adapter tips mounted on a laser handpiece.

FIGS. 5A and 5B are perspective views of a second embodiment of the present invention.

FIGS. 6(A, B, C) to 9 illustrate the internal structure of the second embodiment of the present invention employing a fluid cooling system.

FIGS. 10 to 12 illustrate the internal structure of the second embodiment of the present invention employing a thermoelectric cooling system.

### DETAILED DESCRIPTION OF THE PRESENT INVENTION

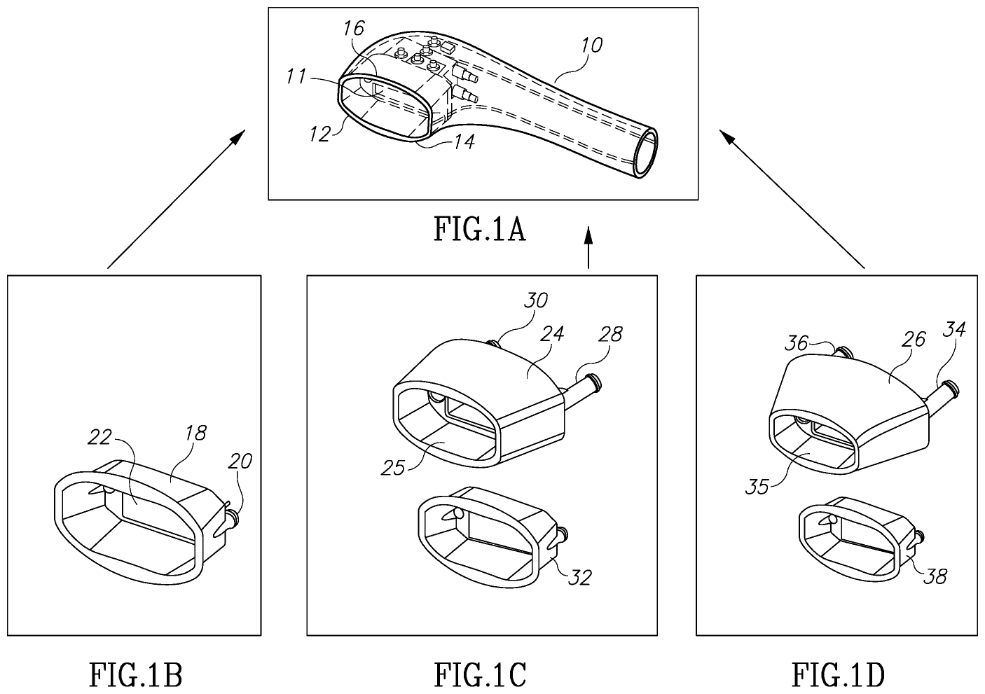

Turning now to FIGS. 1A-1D, FIG. 1A illustrates the present Lightsheer® HS device 10 discussed above. The HS includes a cavity 12 into which the tissue is drawn under the influence of a vacuum which is connected to a vacuum source (not shown). The cavity 12 includes interior walls 14 and at the bottom of the cavity 16 is located the laser diode array 11. In order to provide a clean and sanitary treatment, a disposable tip 18, shown in FIG. 1B, is inserted into the cavity. The disposable tip 18 also includes two tubes (one shown as 20, the other not shown) that connect to the source of vacuum. The bottom of the tip 22 is open or at least of a transparent material so that light from the laser diode array 11 at the bottom of the cavity may be in a position to radiate the skin tissue with, in this device, laser light. However, this device may also include another type of light source, such as a known IPL light source. After a treatment, the disposable tip may be removed from the cavity and discarded. FIGS. 1C and 1D illustrate, respectively, medium and small size replacement tips 24 and 26. Medium size tip 24 includes two tubes 28 and 30 which, when assembled into the open cavity 12 of FIG. 1A will enter into receiving holes in the bottom of the cavity (not shown) that allow the vacuum source to interact with tissue drawn into cavity 25 of the tip 24. The tip 24 may also include a disposable insert 32 like insert 18 but sized to fit into cavity 25. FIG. 1D shows a similar tip 26 which is sized smaller than tip 24 and includes similar tubes 34 and 36, cavity 35 and disposable tip 38. It is to be noted that each of the disposable tips 18, 24 and 26 include tubes 20, 28, 30, 34, 36 for vacuum communication purposes.

Also, it is noted that while three tips are shown and described, any number or size may be provided depending on the size of the treatment area and/or the degree of concentration of light energy desired. In addition, optical elements, such as one or more lenses, may be incorporated in the bottom of each respective cavity to further direct the light coming from the laser diode array contained in device 10. Further, each of the tips may include some form of identification, such as a RFID tag, QR, or pins that would identify to a controller included in an apparatus to which the device 10 is connected which tip is connected and mounted on the handpiece. This is for the purposes of, as and if needed, changing the treatment regimen depending on the size of the tip installed in the cavity. For example, a particular size tip, when inserted and recognized by a programmable controller operatively connected to the tip (through the RFID tag or otherwise), may cause the controller to apply a particular treatment regimen appropriate to that tip.

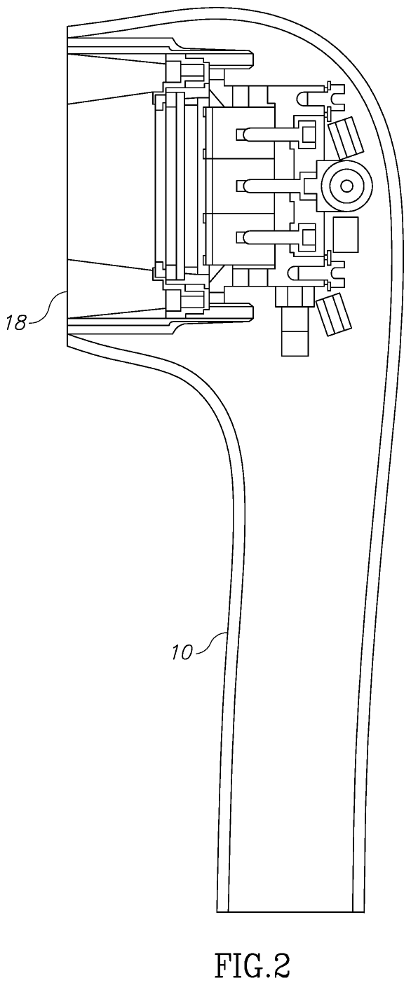

Turning now to the other drawings, FIG. 2 shows the device 10 of FIG. 1A in a larger scale with the disposable tip 18 installed.

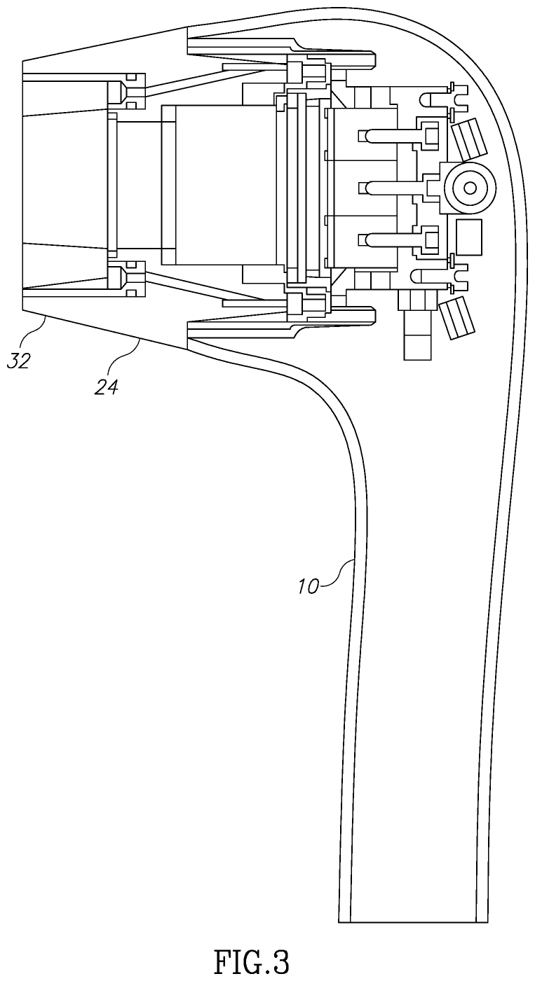

FIG. 3 shows the device 10 of FIG. 1A but with the tip 24 (from FIG. 1C) installed in the cavity 12 ready for use including its respective disposable tip 32.

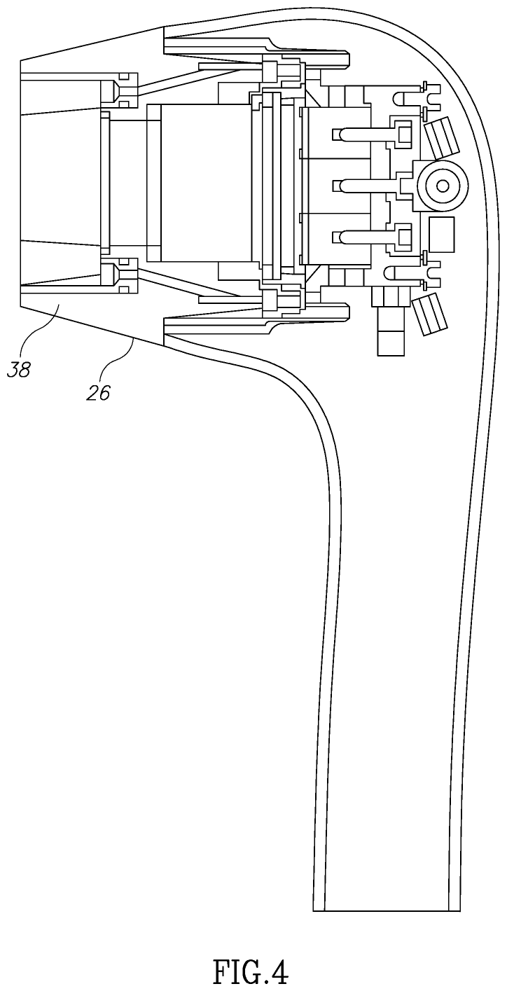

FIG. 4 illustrates the device 10 with the small size tip 26 (from FIG. 1D) installed with its disposable tip 38.

It is envisioned that the above arrangement may be applicable to not only the referenced Lightsheer® device but more generically applicable to handsets that operate at wavelengths different from that of the Lightsheer® device. In addition, while the above description has been of a “standard” HS device which accommodates different size inserts within its cavity 25, it is envisioned that different size handpieces may be made, such as the different size tip handpieces shown in FIGS. 1C and 1D. Also, while the adapters shown for example in FIGS. 1C and 1D retain an open cavity structure, it is further envisioned that vacuum chill tips such as those disclosed in a second embodiment of the present invention, to be discussed below in detail, may be made as adapters of different sizes that will fit into the cavity 12 of FIG. 1A. Finally, while the adapters of the present invention have been shown as fitted into the handpiece cavity 12, it is envisioned that the adapters of FIGS. 1C and 1D may be structured as attachments to a handpiece that does not have in its basic structure a cavity at all but rather the adapters of FIGS. 1C and 1D (and any other sized adapter) may mate with a flat or otherwise non-cavity surface of a handpiece.

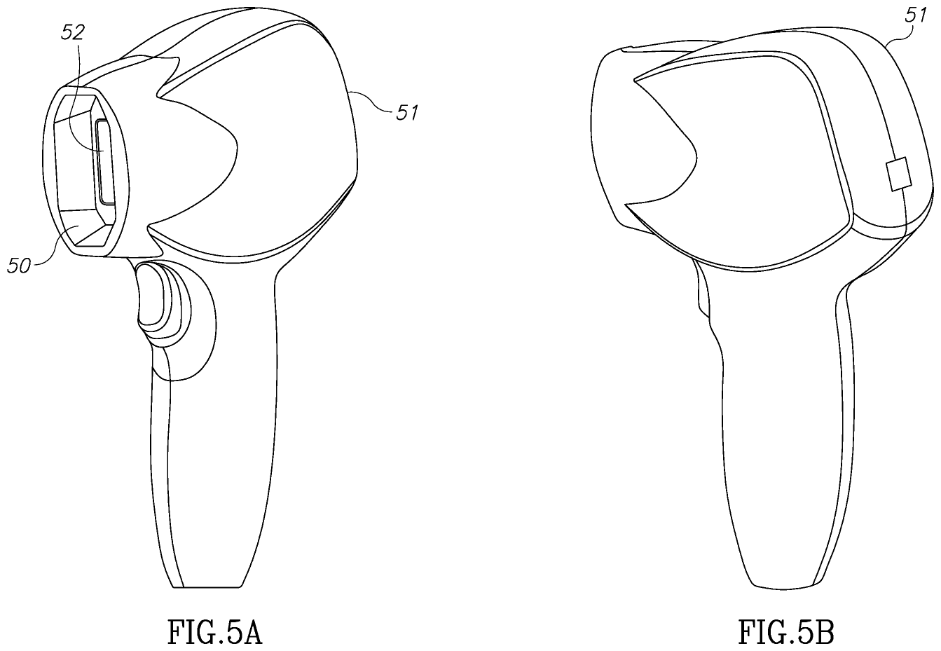

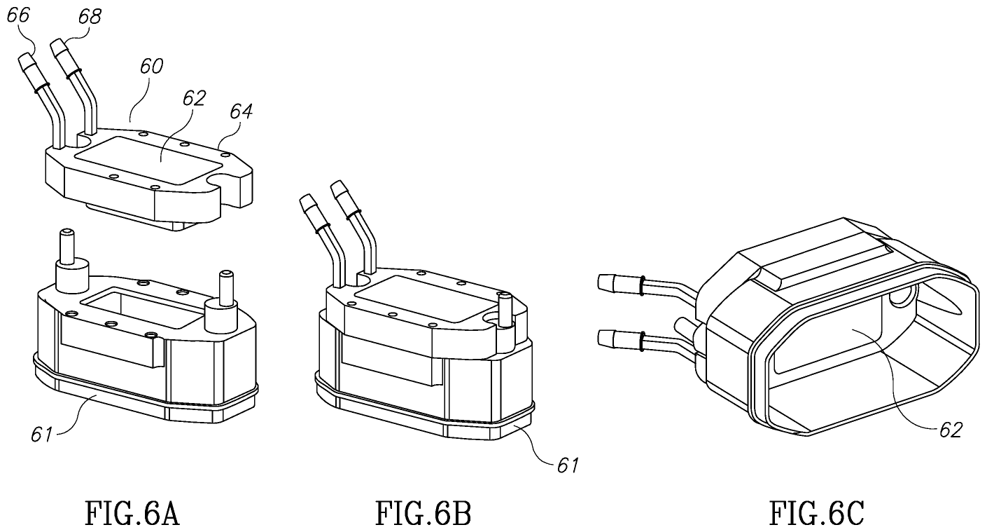

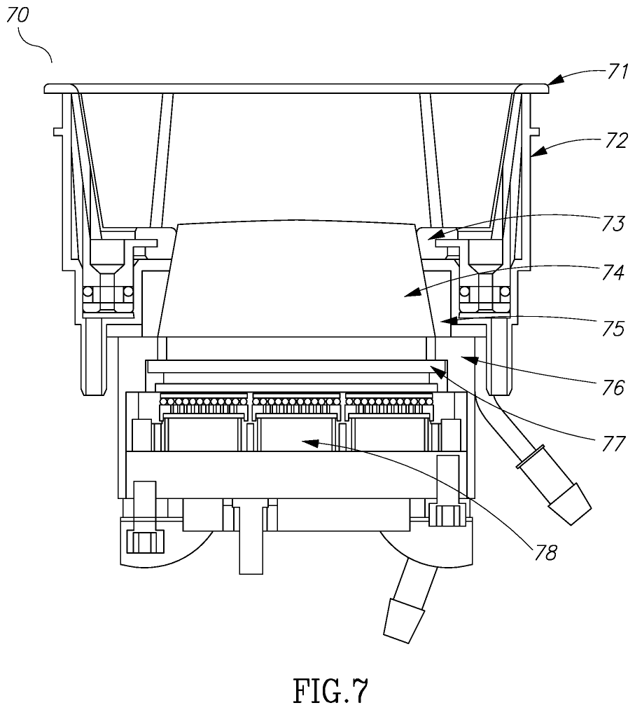

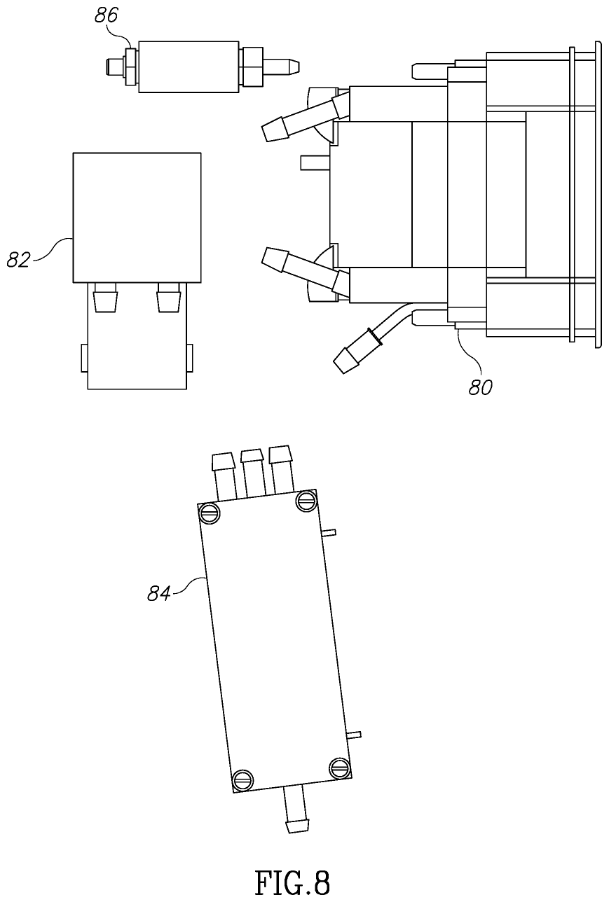

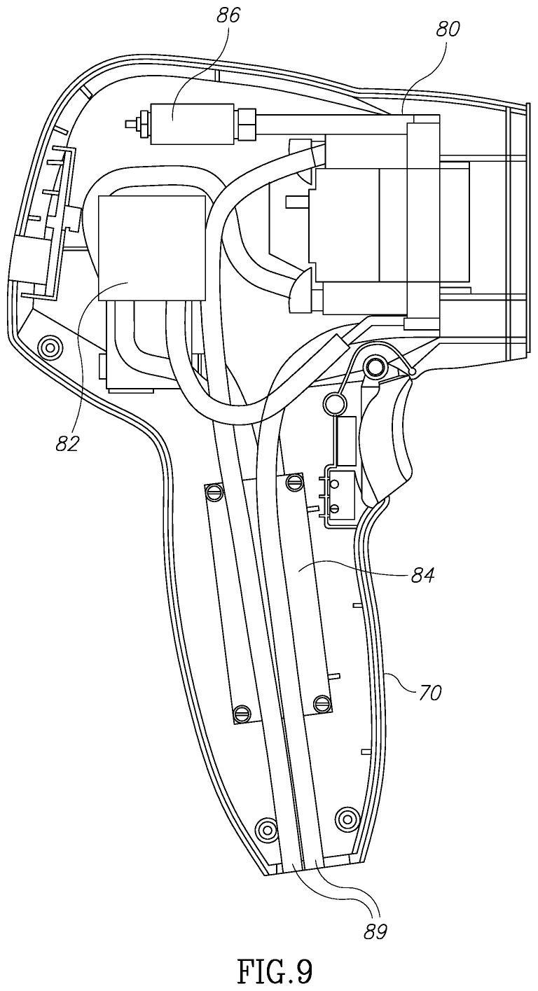

Turning now to a second embodiment of the present invention, FIGS. 5A and 5B provide two perspective views of the present invention handpiece 51. As can be seen in the figure, the handpiece includes a cavity 50 into which, like the HS, the tissue is drawn into under a vacuum force provided in the handpiece. However, unlike the handpiece 10 illustrated in FIG. 1A, the cavity 50 includes a sapphire or other transparent solid or hollow medium 52, to be explained in further detail below with reference to FIGS. 6A to 6C.

FIGS. 6A to 6C show three perspective views of the sapphire assembly 60 and the cavity structure 61, including the sapphire 62 within a surrounding cooling housing 64, liquid coolant inlet 66 and outlet 68. As can be seen, the sapphire is surrounded by the jacket 64 which is open at the bottom and the top to allow positioning of the sapphire 32. Cooling fluid enters through inlet 66, passes through jacket 64, thus cooling the sapphire and then exits through outlet 68 to be cooled again and returned. FIG. 6B shows the sapphire assembly mounted on the cavity structure 61 and FIG. 6C provides an inside view of the assembly, showing the sapphire 62.

Turning now to FIG. 7, this figure shows the full assemblage 70 of one embodiment of the device of the present invention. The assemblage 70 includes, in order from the top of the figure to the bottom of the figure, a disposable tip 71 (like those discussed in connection with FIGS. 1 through 4) that is fitted into the tip housing or cavity assembly 72, a gasket 73 surrounding the sapphire 74, an EPI cooler 75, thermal isolation 77 that on one side isolates the sapphire from the diode array 78 from being overheated from the diode array light bars 78 but on the other side does not decrease the temp near the bars to avoid condensation on the light bars and a section of protective glass 77 to protect the light bars of the diode array 78.

FIG. 8 once again shows the assemblage 80 discussed in conjunction with FIG. 7 with additional parts to aid in the cooling function including a fluid cooling pump 82 that moves cooling fluid through a heat exchanger 84 into the assemblage 80 to cool both the sapphire and the light diode assembly as well as vacuum sensor 86 which senses the presence and/or level of vacuum with the cavity during operation.

FIG. 9 illustrates further details the mounting of the components of FIG. 8 in the handpiece 70 together with tubes or conduits 89 that connect the components and carry cooling fluid into and out of the handpiece 70.

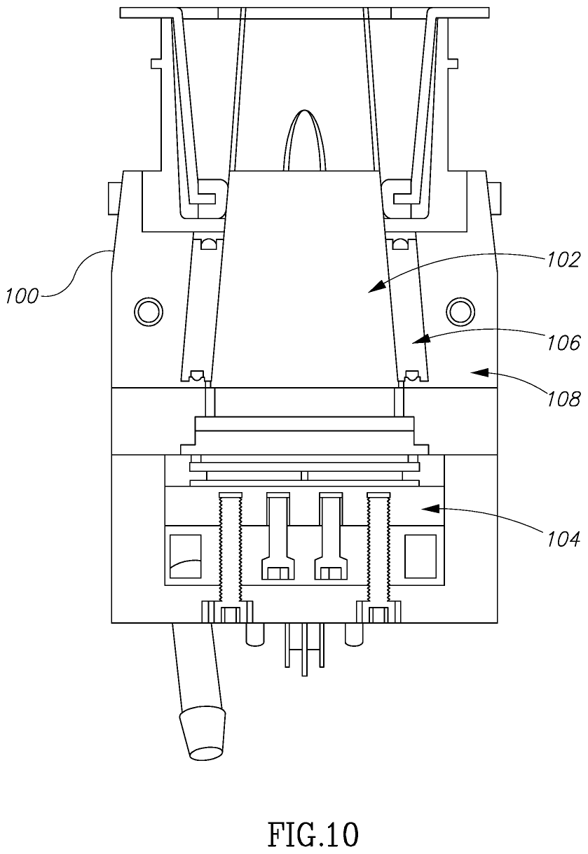

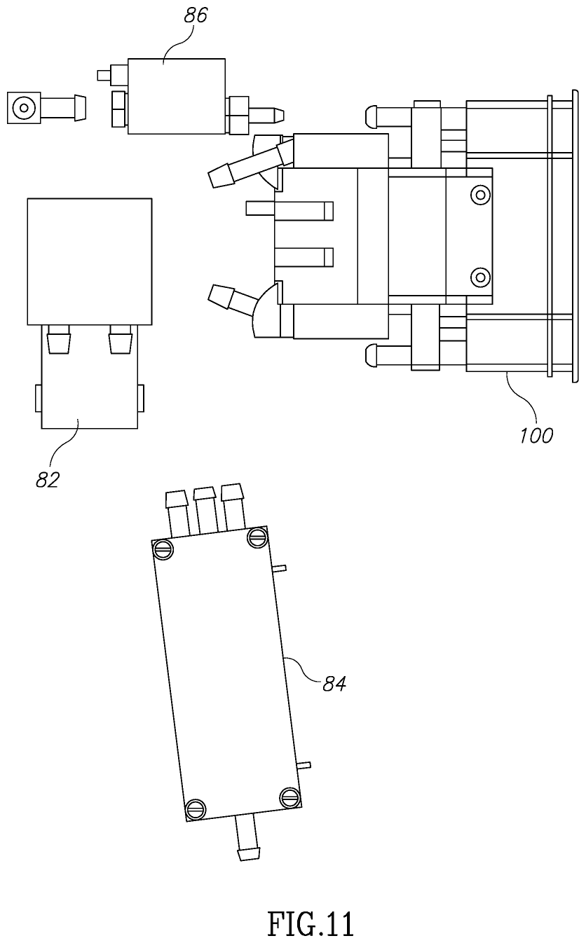

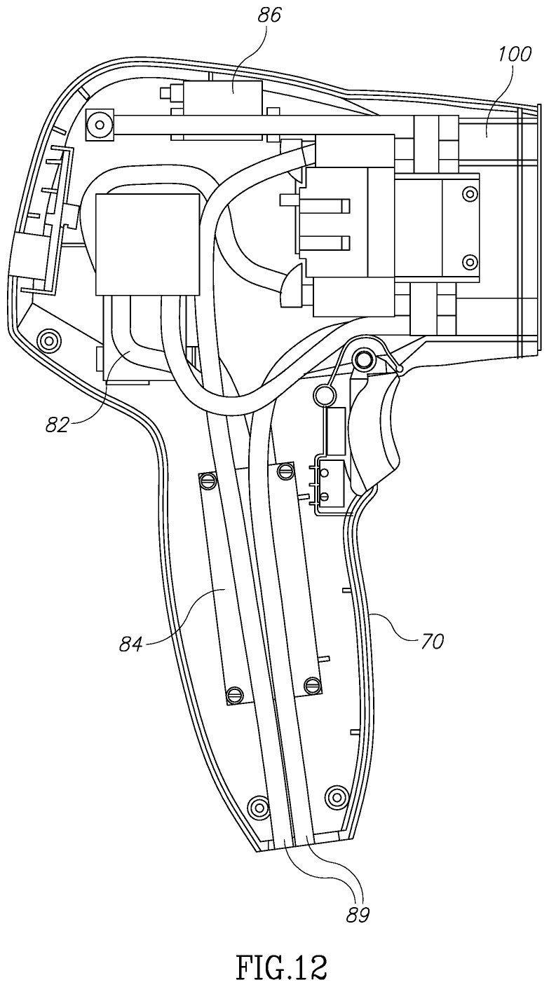

FIG. 10 provides details of an alternative arrangement of an embodiment 100 to cool the sapphire 102 and the diode array 104 in front and back perspective view. Unlike FIG. 7, FIG. 10 does not include fluid cooling for the sapphire but rather substitutes one or more thermoelectric coolers (TEC) 106 that may surround and may contact the sapphire 102. A cooling block collar 108 may be provided that helps to maintain the cooling of the sapphire by acting as a heat sink. FIG. 11 is similar to FIG. 8 and illustrates the vacuum sensor 86 and the fluid cooling components 82 and 84 that are provided to cool the diode assembly while the TEC cooler cools the sapphire itself. The cooling fluid also may remove heat generated from the non-cooling side of the TEC while the cooling side of the TEC cools the sapphire. Of course, it is within the scope of the present invention that the diode array and/or the sapphire may be cooled by a combination of cooling fluid and TEC cooling.

FIG. 12 is a figure similar in scope to FIG. 9 and shows the placement of components from FIG. 11 into a handpiece, including tubes or passages 89.

Thus, with the present invention, the benefits of both the Lightsheer® HS and the ET are combined in a way so that large areas of skin tissue can be treated within a vacuum environment but with the additional benefit of cooling to provide a safer treatment. The present invention is not just a larger sized ET, but rather adds the benefit of vacuum in that when the skin is drawn into the cavity, blood within the tissue is “pushed out” of the volume within the cavity, thus allowing a better treatment regimen the absence of blood being impinged upon by the light from the laser diodes.

Thus, in operation, an operator may place the handpiece, for example, the handpiece 51 shown in FIG. 5A, on to the skin tissue of a patient to be treated. After placing in contact, a vacuum source may be activated to draw the skin tissue into the cavity 50. Once the tissue is in place within the cavity, the tissue is ready for treatment. Prior to or simultaneous with or even after the diode array is fired, the cooling mechanism(s) described above may be activated and the sapphire cooled so that the skin tissue is cooled while the treatment takes place or even afterwards.

1. An adapter tip for a light emitting device, the light emitting device having a cavity, the cavity being open on one end of size X, depending side walls beginning at the open end and extending to a closed bottom wall, wherein the closed bottom wall includes a light source which projects light from the closed bottom wall to and out the open end, the adapter comprising:
a housing having a plurality of joined side walls, the side walls terminating at two open ends;
one of the open ends being sized at about the same size X as the open end of the cavity and attachable to the open end of the cavity;
the other of the open ends being tapered from size X to a size Y, wherein Y is less than X in size;
whereby light projected from the light source is reduced from size X to size Y when emitted from the other of the open ends.

2. The adapter tip of claim 1, the light emitting device further comprising one or more apertures formed on the closed bottom wall, the one or more apertures being connectable to a vacuum source,
the adapter further comprising one or more tubes mounted on the open end of the adapter of about size X, the one or more tubes being insertable into the one or more apertures formed on the closed bottom wall, the one or more tubes operating to join the adapter to the light emitting device.

3. The adapter of claim 1, further comprising a disposable insert adapted for insertion into the other of the open ends in the adapter.

4. The adapter of claim 1, further comprising a plurality of adapters and wherein the other of the open ends in the plurality of adapters are of size Z, wherein Z is less than size Y.

5. A light emitting device for treating skin tissue, the light emitting device having a housing, the housing having a cavity, the cavity being open on one end, a plurality depending side walls beginning at the open end and extending to a closed bottom wall, wherein the closed bottom wall includes a light source which projects light from the closed bottom wall to and out the open end;
a solid transparent member mounted within the cavity, the member having two opposed faces and a plurality of side walls joining the two opposed faces;
the member being positioned in the cavity distally of the light source;
a collar mounted on and surrounding the plurality of side walls of the solid transparent member, the collar being constructed of a thermally conductive material;
a cooling source operatively connected to the collar, the cooling source cooling the collar and the surrounded solid transparent member;
a vacuum source, the vacuum source being operatively connected to one or more formed openings in the cavity;
the vacuum source, when activated, drawing skin tissue into the cavity to the extent that the skin tissue contacts the cooled solid transparent member;
whereby, upon activation of the light source, heat is emitted from the light source, the skin tissue being cooled by the cooled solid transparent member.

6. The light emitting device of claim 5, wherein the light source is one or more arrays of laser diodes.

7. The light emitting device of claim 5, wherein the cooling source is a cooled fluid, the collar including passages through which the cooled fluid enters and leaves the collar.

8. The light emitting device of claim 5, wherein the cooling source is a thermoelectric cooling device, the thermoelectric cooling device being mounted between the solid transparent member and the surrounding collar, further comprising one or more passages in the collar through which cooling fluid enters and leaves the collar.

9. A method of cosmetically treating the skin tissue, the steps comprising:
providing the light emitting device according to claim 5;
placing the open end of the device onto the skin tissue;
activating the vacuum source to thereby draw skin to draw the skin tissue into the cavity towards and in contact with a face of the solid transparent member;
activating the cooling source to cool the solid transparent member; activating the light source, the light source causing light to travel from the light source through to the solid transparent member and to impinge on the skin tissue;
whereby the skin tissue is cooled by contact with the solid transparent member.
